(19) United States
(12) Patent Application Publication
(10) Pub. No.: US 2021/0139518 A1
(43) Pub. Date: May 13, 2021

(54) NOVEL POLYMORPHIC FORM OF FERRIC MALTOL

(71) Applicant: RK Pharma Solutions LLC, Piscataway, NJ (US)

(72) Inventors: Ravishanker Kovi, Monroe Township, NJ (US); Jayaraman Kannappan, Vadodara (IN); Ananda Babu Thirunavakarasu, Vadodara (IN); Hemant Mande, Vadodara (IN); Rajesh A. Patel, Vadodara (IN)

(21) Appl. No.: 17/079,554

(22) Filed: Oct. 26, 2020

(30) Foreign Application Priority Data
Oct. 26, 2019 (IN) 201921043623

(51) Int. Cl. C07F 15/02; C07D 309/40

(52) U.S. Cl. CPC C07F 15/025; C07B 2200/13; C07D 309/40

(57) ABSTRACT

The present application generally provides ferric maltol of formula I, and more particularly a crystalline form alfa of Ferric maltol, which is characterized by its PXRD pattern as illustrated herein. Corresponding methods for producing the ferric maltol disclosed herein are also provided.

### FIELD OF THE INVENTION

The present application relates to Ferric maltol of formula I. Specifically, the present application relates to novel crystalline form alfa of Ferric maltol and process for the preparation of such novel polymorph. The application is further directed to pharmaceutical compositions comprising of novel polymorphic form of Ferric maltol. The application also present the use of this novel pharmaceutical form containing Ferric maltol composition for the treatment of iron deficiency and related diseases.

### BACKGROUND OF THE INVENTION

ACCRUFER® (ferric maltol) is used as an iron replacement product for oral administration. Accrufer® is available in capsule form which contains 30 mg iron and 201.5 mg of maltol. Ferric maltol contains iron in a stable ferric state as a complex with a trimaltol ligand. Ferric maltol is 3-hydroxy-2-methyl-4H-pyrane-4-one iron (III) complex (3:1) and has the molecular formula (C₆H₅O₃)₃Fe and a molecular mass of 431.2 g/mol. The structure of ferric maltol is represented in formula I.

[chemical structure]

Each red capsule, printed with “30”, contains colloidal anhydrous silica, crospovidone (Type A), lactose monohydrate, magnesium stearate and sodium lauryl sulfate as inactive ingredients. In addition, the capsule shell contains FD&C Blue No. 1, FD&C Red No. 40, FD&C Yellow No.6, gelatin and titanium dioxide. The ink used for printing the marking contains ammonium hydroxide, ethanol, iron oxide black and propylene glycol.

U.S. Pat. Nos. 6,339,080 and 6,63,563, which are incorporated herein by reference, discusses the formation of iron complexes of 3-hydroxy-4-pyrones where a carboxylic acid is provided as a counterion. EP 0159194, which is also incorporated herein by reference, discusses the neutral (i.e., charge balanced) ferric iron complexes comprising specified combinations of ligands selected from 3-hydroxypyrones, 3-hydroxypyridones and specific mono-carboxylic acids. In order to produce neutral (i.e., charge balanced) ferric complexes, EP 0159194 discusses the reaction of an ethanolic solution of ferric chloride with a chloroform solution of a hydroxypyrone ligand followed by adjustment of the pH with solid sodium carbonate to synthesis ferric compound.

GB 2128998, GB 2157563, and EP 0107458, which are also incorporated herein by reference, discuss the method of preparation of neutral (i.e., charge balanced) iron (III) maltol complexes which involves mixing a solution of maltol in chloroform with a 1M solution of ferric chloride in ethanol to provide a 3:1 molar ratio of maltol:iron in the mixture.

GB 2136806, which is also incorporated herein by reference, discusses the preparation of an iron hydroxypyridone complex using freeze drying and organic solvents. WO 2003097627, which is also incorporated herein by reference, discusses the method of forming an iron hydroxypyrone compound comprising reacting an iron salt of a carboxylic acid and a hydroxypyrone in an aqueous solution at a pH greater than 7. Nurchi et al (Journal of Inorganic Biochemistry, 104, 2010, 560-569), which is also incorporated herein by reference, discusses the synthesis of a ferric tri-kojic acid chelate which is very soluble in the solution and so does not precipitate.

Polymorphism, the occurrence of different crystal form is a property of some molecules and molecular complexes. A single molecule, like Ferric maltol, may rise to variety of crystalline forms having distinct crystal structures and physical properties like melting point, X-ray diffraction pattern, infra absorption fingerprint, and solid state NMR spectrum. One crystalline form may give rise to thermal behaviour different from that of another crystalline form.

Different salts and solid state forms (including solvated forms) of an active pharmaceutical ingredient may possess different properties. Such variations in the properties of different salts and solid state forms and solvates may provide a basis for improving formulation, for example, by facilitating better processing or handling characteristics, improving the dissolution profile, or improving stability (polymorph as well as chemical stability) and shelf-life. These variations in the properties of different salts and solid state forms may also provide improvements to the final dosage form, for instance, if they serve to improve bioavailability. Different salts and solid state forms and solvates of an active pharmaceutical ingredient may also give rise to a variety of polymorphs or crystalline forms, which may in turn provide additional opportunities to use variations in the properties and characteristics of a solid active pharmaceutical ingredient for providing an improved product.

Discovering new salts, solid state forms and solvates of a pharmaceutical product can provide materials having desirable processing properties, such as ease of handling, ease of processing, storage stability, and ease of purification or as desirable intermediate crystal forms that facilitate conversion to other salts or polymorphic forms.

In particular compound ferric maltol several methods are known to synthesis the ferric maltol but very limited literature has disclosed the polymorphic forms of this ferric maltol.

U.S. Pat. No. 9,802,973, which is also incorporated herein by reference, has claimed four different forms of ferric maltol. It further discusses the process for the preparation of these form. Form disclose in the patents are form I, form II, form III and form IV. Same patent further covers the use of these polymorphic forms containing ferric maltol for the preparation of pharmaceutical composition.

Since improved drug formulations are consistently sought, there is an ongoing need for new or purer polymorphic form of existing drug molecules. For at least these reasons, there is a need for additional solid state forms (including solvated forms) of ferric maltol.

The present application describes novel crystalline form alfa of Ferric maltol of formula I and its process for the preparation thereof.

### SUMMARY OF THE INVENTION

In a first embodiment, the present application provides Ferric maltol of formula I.

In a second embodiment, the present application provides crystalline form alfa of Ferric maltol that can be characterized by its PXRD pattern as illustrated by FIG. 1.

In a third embodiment, the present application provides a process for preparing a crystalline form alfa of Ferric maltol, which involves one or more of the following steps:

a) providing a solution of Ferric maltol in an organic solvent;

b) evaporating the solvent at higher temperature; and

c) isolating form alfa Ferric maltol.

The process is represented in Table A below.

TABLE A

[chemical structure]

[chemical structure]

In a fourth embodiment, the present application provides a pharmaceutical composition comprising a highly pure novel crystalline form alfa of Ferric maltol and one or more pharmaceutically acceptable excipients.

In another embodiment, the present application provides the process for preparation Ferric maltol-technical of formula I.

At least one step involves a reaction of maltol with ferric chloride hexahydrate in water by using sodium hydroxide as base to get ferric maltol technical.

This reaction is represented in Table B below.

TABLE B

[chemical structure]

[chemical structure]

In at least one aspect, a crystalline Ferric maltol form alfa is provided that is characterized by a PXRD pattern having peaks at 9.4, 12.7, 14.4, 15.2, 17.3, 19.8, 21.1, 23, 24.3° 2Θ±0.2° 2Θ, in at one embodiment, as in FIG. 1.

In at least one aspect, a pharmaceutical composition is provided that includes a highly pure crystalline alfa form of Ferric maltol and one or more pharmaceutically acceptable excipients.

In at least one embodiment, the crystalline alfa form of Ferric maltol is characterized as described herein.

In at least one aspect, a process for preparing the crystalline Ferric maltol form alfa is provided that includes dissolving ferric maltol in at least one solvent; evaporating the solvent at a temperature between about 70° C. and about 100° C.; and isolating Ferric maltol form alfa therefrom.

In at least one embodiment, the ferric maltol is dissolved in a polar aprotic solvent selected from the group consisting of dimethylformamide, dimethylacetamide, dimethylsulfoxide, dichloromethane, and ethyl acetate.

In at least one embodiment, the ferric maltol is dissolved in an ether solvent selected from the group consisting of tetrahydrofuran, dioxane, methyl tertiary butyl ether, and diethyl ether.

In at least one embodiment, the ferric maltol is dissolved in a chloro solvent selected from the group consisting of dichloromethane, chloroform, and dichloroethane.

In at least one embodiment, the ferric maltol is dissolved in an alcoholic solvent selected from the group consisting of methanol, ethanol, isopropanol, and butanol.

In at least one aspect, a process for preparing a crystalline form of Ferric maltol of formula I is provided that includes dissolving maltol in water; reacting the maltol solution with ferric chloride hexahydrate in water, in the presence of base sodium hydroxide; adjusting the pH of the solution to about 7 pH; distilling the reaction mixture under vacuum, below 55° C.; dissolving residue in a chloro solvent selected from the group consisting of dichloromethane, chloroform, and dichloroethane; filtering and evaporating the resulting solution under vacuum below 50° C. to obtain ferric maltol.

### BRIEF DESCRIPTION OF THE DRAWINGS

FIG. 1 illustrative a characteristic X-ray powder diffraction pattern of Ferric maltol form alfa.

### DESCRIPTION OF THE INVENTION

Embodiments of the present application now will be described more fully hereinafter with reference to the accompanying examples and experiments, in which illustrative embodiments of the application are shown. The inventions herein may, however, be embodied in many different forms and should not be construed as limited to the embodiments set forth herein; rather, these embodiments are provided so that this disclosure will be thorough and complete, and will fully convey the scope of the application to those skilled in the art. Well-known functions or constructions may not be described in detail for brevity and/or clarity.

The terminology used herein is for the purpose of describing particular embodiments only and is not intended to be limiting. As used herein, the singular forms “a”, “an” and “the” are intended to include the plural forms as well, unless the context clearly indicates otherwise. Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

All percentages and ratios used herein are by weight of the total composition and all measurements made are at 25° C. and normal pressure unless otherwise designated. All temperatures are in degrees Celsius unless specified otherwise.

As used herein, “comprising” means the elements recited, or their equivalent in structure or function, plus any other element or elements which are not recited. The terms “having” and “including” are also to be construed as open ended unless the context suggests otherwise.

All ranges recited herein include the endpoints, including those that recite a range “between” two values.

In a first embodiment, the present application provides Ferric maltol of formula I.

In a second embodiment, the present application provides crystalline form alfa of Ferric maltol that can be characterized by its PXRD pattern as illustrated by FIG. 1.

In a third embodiments, the present application provides process for preparing a Ferric maltol form alfa, comprising steps of:

a) providing a solution of Ferric maltol in a solvent;

b) evaporating the solvent at higher temperature; and

c) isolating form alfa of Ferric maltol.

The process is represented in Table A herein.

Providing a solution in step a) may include:

1) direct use of a reaction mixture containing Ferric maltol, this may be obtained in the course of its synthesis; or

2) direct use of reaction mixture containing Ferric maltol that may be obtained by treating source of ferrous with maltol; or

3) dissolving the ferric maltol in a solvent.

Any physical form of ferric maltol may be utilized in step (a) of the process embodiments herein above.

Suitable solvents which can be used in step (a) for the preparation of form alfa Ferric maltol includes halogenated solvent such as dichloromethane, dichloroethane, chloroform, carbon tetrachloride, 1,2-dichlorobenzene and so like.

The solution obtained in step (a) may be filtered to remove any insoluble particles. Suitable techniques to remove insoluble particles are filtration, micron filter, centrifugation, decantation, and any other techniques known in the art. The solution can be filtered by passing through paper, or other membrane material, or a clarifying agent such as celite. Depending upon the equipment used and the concentration and temperature of the solution, the filtration apparatus may need to be preheated to avoid premature precipitation of solid.

Step (b) may involve evaporating the solvent at higher temperature. The evaporation of solution containing Ferric maltol may be induced by using conventional techniques known in the art. For example, useful techniques include but are not limited to heating at higher temperature then boiling temperature of solvent, flash evaporation, simple evaporation and so like other techniques.

The solvent can be removed, optionally under reduced pressures, at temperatures less than about 100° C., less than about 80° C., less than about 60° C. or any other suitable temperatures.

Step (c) involves isolation of form alfa Ferric maltol from reaction vessel. The isolation of crystalline form alfa of Ferric maltol may be induced by using conventional techniques known in the art. For example, useful techniques include but are not limited to concentrating, cooling, stirring, scraping, shaking, combining with an anti-solvent, adding seed crystals, evaporation, flash evaporation, simple evaporation, rotational drying, or the like. The solid that is obtained may carry a small proportion of occluded mother liquor containing a higher percentage of impurities and, if desired, the solid may be washed with a solvent to wash out the mother liquor. Evaporation as used herein refers to distilling of solvent almost completely at atmospheric pressure or under reduced pressure. Flash evaporation as used herein refers to distilling of solvent by using a technique includes but is not limited to tray drying, fluidized bed drying. The recovery of crystalline form alfa can be done by decantation, centrifugation, gravity filtration, suction filtration and like.

Particularly, crystalline forms may also be obtained by heating or melting a form obtained followed by gradual or fast cooling; in this manner one polymorph or one crystalline form may be converted to another.

In another aspect present application involves recovery of crystalline Ferric maltol after removal of solvent. The said recovery can be done by using the processes known in the art. The resulting solid may be collected by using techniques such as by scraping, or by shaking the container, or other techniques specific to the equipment used. The isolated solid may be optionally further dried to afford crystalline Ferric maltol.

The resulting compound may be optionally further dried. Drying can be carried out in a tray dryer, vacuum oven, air oven, buchi®, rotavapor®, cone vacuum dryer, rotary vacuum dryer, fluidized bed dryer, spin flash dryer, flash dryer, gravity oven, or the like. The drying can be carried out at temperatures of less than about 120° C., less than about 100° C., less than about 80° C., less than about 60° C., or any other suitable temperatures; at atmospheric pressure or under a reduced pressure; as long as the crystalline Ferric maltol is not degraded in its quality. The drying can be carried out for any desired times until the required product quality is achieved. Suitable time for drying can vary from few minutes to several hours for example from about 30 minutes to about 24 or more hours.

Once obtained, crystals of Ferric maltol form alfa may be used as the nucleating agent or “seed” crystals for subsequent crystallizations of Ferric maltol from solutions.

The crystalline form of Ferric maltol form alfa herein have advantageous properties selected from at least one of: chemical purity, stability - such as storage stability, stability to dehydration, stability to polymorphic conversion, flowability, solubility, morphology or crystal habit, low hygroscopicity and low content of residual solvents.

In a fourth embodiment, the present application provides pharmaceutical formulations comprising Ferric maltol of form alfa with one or more pharmaceutically acceptable excipients. The excipients of the present application may be formulated as: solid oral dosage forms such as, but not limited to, powders, granules, pellets, tablets, and capsules; liquid oral dosage forms such as, but not limited to, syrups, suspensions, dispersions, and emulsions; and injectable preparations such as, but not limited to, solutions, dispersions, and freeze dried compositions. Formulations may be in the forms of immediate release, delayed release, or modified release. Further, immediate release compositions may be conventional, dispersible, chewable, mouth dissolving, or flash melt preparations, and modified release compositions that may comprise hydrophilic or hydrophobic, or combinations of hydrophilic and hydrophobic, release rate controlling substances to form matrix or reservoir or combination of matrix and reservoir systems. The compositions may be prepared using any one or more of techniques such as direct blending, dry granulation, wet granulation, and extrusion and spheronization. Compositions may be presented as uncoated, film coated, sugar coated, powder coated, enteric coated, and modified release coated.

Pharmaceutically acceptable excipients that are useful in the present application are the same as defined above.

The pharmaceutical dosage form according to the present application may be coated with one or more coating materials or uncoated. The coating materials are not particularly limited and are known to the person skilled in the art.

The pharmaceutical dosage form according to the present application can further comprise additional excipients and adjuvants, which are pharmaceutically acceptable and general coating materials, which are preferably applied as a coating to the pharmaceutical dosage form of the present application. Such further excipients and adjuvants are known to the person skilled in the art.

In another embodiment, present application provides the process for preparation Ferric maltol-technical of formula I.

Process involves a reaction of maltol with ferric chloride hexahydrate in water by using sodium hydroxide as base to get ferric maltol technical.

The reaction is represented in table B herein.

Certain specific aspects and embodiments of the present application will be explained in more detail with reference to the following examples, which are provided only for purposes of illustration and should not be construed as limiting the scope of the present application in any manner.

### EXAMPLES

### Example 1

### Preparation of Ferric Maltol Technical

1 gm of maltol was dissolved into 30 ml water at RT. Solution containing 0.69 gm ferric chloride hexahydrate in 10 ml of water was slowly added in above solution at RT. pH of the solution was adjusted to around 7 by using sodium hydroxide solution (0.16 gm in 3 ml of water). Reaction mixture was stirred for 30 minutes. Reaction mixture was distilled under vacuum below 55° C. Dichloromethane 10 ml was charged in above residue. The solution is filtered to remove undissolved particulate matters. The filtered solution then completely evaporated under vacuum below 50° C. to get Ferric maltol technical.

Yield: 1.3-1.7 g

### Example 2

### Preparation of Ferric Maltol Form Alfa

0.5g of ferric maltol was dissolved into 5 ml dichloromethane at RT. The solution was filtered to remove undissolved particulate. The filtered solution then completely evaporated on rota vapour at 90° C. The precipitated material was dried at 90° C. for 2-3h to yield Ferric maltol having polymorph form alfa.

Yield: 0.48 g

While the foregoing invention has been described in some detail for purposes of clarity and understanding, it will be appreciated by one skilled in the art, from a reading of the disclosure, that various changes in form and detail can be made without departing from the true scope of the invention.

What is claimed is:

1. A crystalline Ferric maltol alfa form characterized by a PXRD pattern having peaks at 9.4, 12.7, 14.4, 15.2, 17.3, 19.8, 21.1, 23, 24.3° 2Θ±0.2° 2Θ.

2. The crystalline Ferric maltol alfa form of claim 1, characterized by a PXRD pattern as in FIG. 1.

3. A pharmaceutical composition, comprising a highly pure crystalline alfa form of Ferric maltol and one or more pharmaceutically acceptable excipients.

4. The composition of claim 3, wherein the crystalline alfa form of Ferric maltol is characterized as in claim 1.

5. A process for preparing the crystalline Ferric maltol alfa of claim 1, comprising:
a) dissolving ferric maltol in at least one solvent;
b) evaporating the solvent at a temperature between about 70° C. and about 100° C.; and
c) isolating alfa Ferric maltol form therefrom.

6. The process of claim 5, wherein the ferric maltol is dissolved in a polar aprotic solvent selected from the group consisting of dimethylformamide, dimethylacetamide, dimethylsulfoxide, dichloromethane, and ethyl acetate.

7. The process of claim 5, wherein the ferric maltol is dissolved in an ether solvent selected from the group consisting of tetrahydrofuran, dioxane, methyl tertiary butyl ether, and diethyl ether.

8. The process of claim 5, wherein the ferric maltol is dissolved in a chloro solvent selected from the group consisting of dichloromethane, chloroform, and dichloroethane.

9. The process of claim 5, wherein the ferric maltol is dissolved in an alcoholic solvent selected from the group consisting of methanol, ethanol, isopropanol, and butanol.

10. A process for preparing a crystalline form of Ferric maltol of formula I comprising:
a) dissolving maltol in water;
b) reacting the maltol solution with ferric chloride hexahydrate in water, in the presence of base sodium hydroxide;
c) adjusting the pH of the solution to about 7 pH;
d) distilling the reaction mixture under vacuum, below 55° C.;
e) dissolving residue in a chloro solvent selected from the group consisting of dichloromethane, chloroform, and dichloroethane;
f) filtering and evaporating the resulting solution under vacuum below 50° C. to obtain ferric maltol.
